(19) United States
(12) Patent Application Publication
(10) Pub. No.: US 2019/0350565 A1
(43) Pub. Date: Nov. 21, 2019

(54) FEMALE URINE COLLECTION ASSEMBLY

(71) Applicant: Patricia Hiddleson, West Chester, PA (US)

(72) Inventor: Patricia Hiddleson, West Chester, PA (US)

(21) Appl. No.: 15/985,124

(22) Filed: May 21, 2018

(51) Int. Cl. A61B 10/00; A61F 5/455

(52) U.S. Cl. CPC A61B 10/007; A61F 5/455

(57) ABSTRACT

A female urine collection assembly for collecting a urine sample from a female includes a funnel that has a top end and a bottom end. The top end tapers between a narrow side and a wide side such that the top end has an ovoid shape. In this way the top end may conform to female genitalia thereby facilitating the top end to capture urine for a urine sample. A jar is provided and the funnel is fluidly coupled to the jar thereby facilitating the jar to receive the urine for the urine sample.

### CROSS-REFERENCE TO RELATED APPLICATIONS

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to urine collection devices and more particularly pertains to a new urine collection device for collecting a urine sample from a female.

### BRIEF SUMMARY OF THE INVENTION

An embodiment of the disclosure meets the needs presented above by generally comprising a funnel that has a top end and a bottom end. The top end tapers between a narrow side and a wide side such that the top end has an ovoid shape. In this way the top end may conform to female genitalia thereby facilitating the top end to capture urine for a urine sample. A jar is provided and the funnel is fluidly coupled to the jar thereby facilitating the jar to receive the urine for the urine sample.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a female urine collection assembly according to an embodiment of the disclosure.

FIG. 2 is a bottom view of a funnel of an embodiment of the disclosure.

FIG. 3 is an exploded perspective view of an embodiment of the disclosure.

FIG. 4 is a left side perspective view of an embodiment of the disclosure.

FIG. 5 is a perspective in-use view of an embodiment of the disclosure.

FIG. 6 is a perspective view of an embodiment of the disclosure showing a cap being coupled to a jar.

FIG. 7 is a cross sectional view taken along line 7-7 of FIG. 4 of an embodiment of the disclosure.

### DETAILED DESCRIPTION OF THE INVENTION

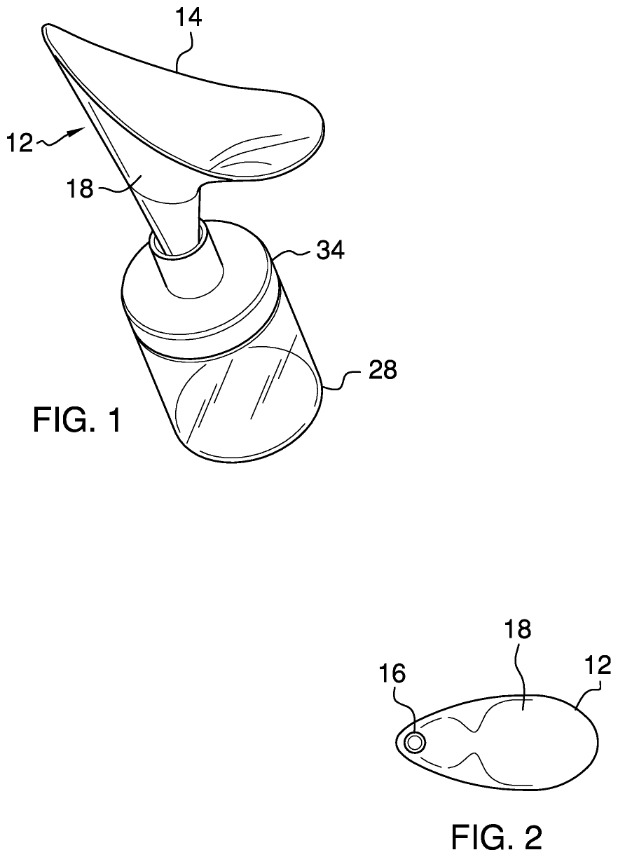

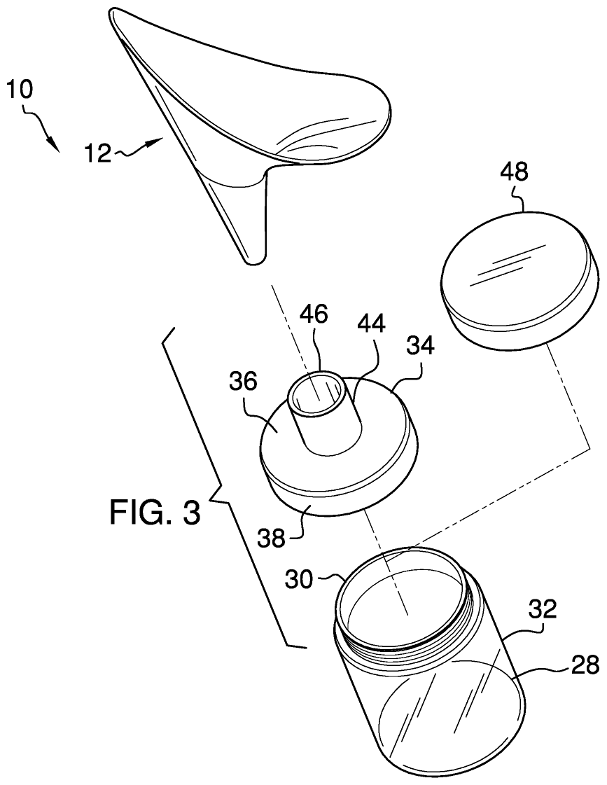

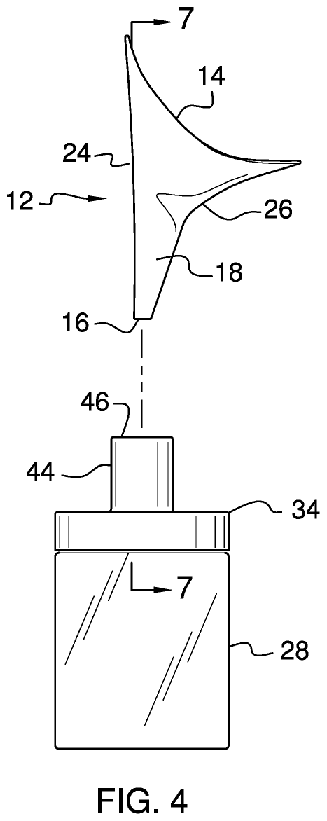

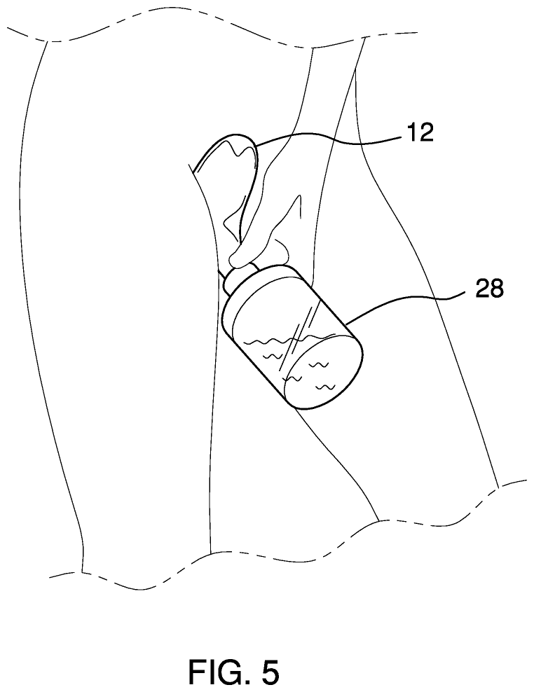

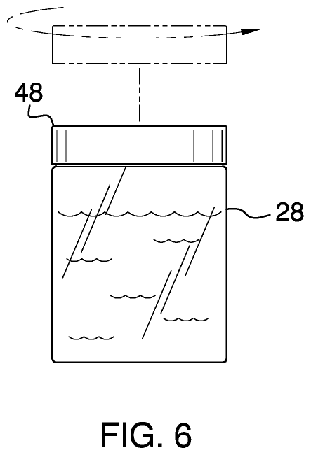

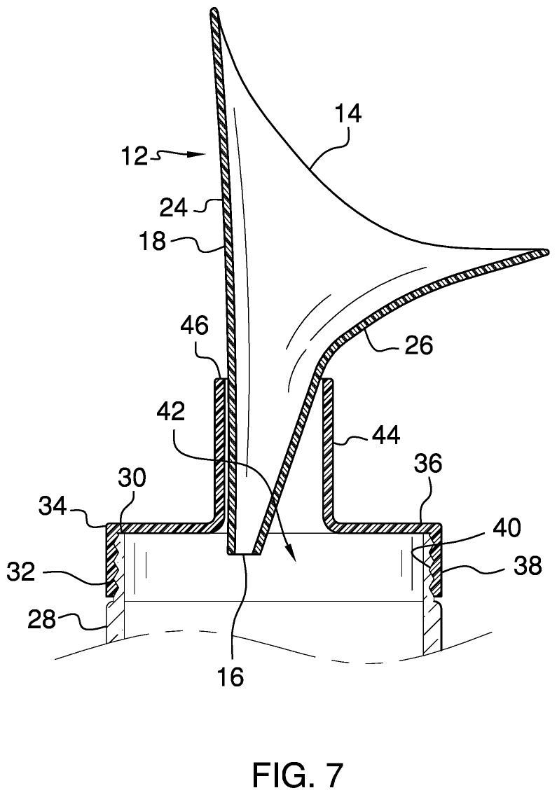

With reference now to the drawings, and in particular to FIGS. 1 through 7 thereof, a new urine collection device embodying the principles and concepts of an embodiment of the disclosure and generally designated by the reference numeral 10 will be described.

As best illustrated in FIGS. 1 through 7, the female urine collection assembly 10 generally comprises a funnel 12 that has a top end 14, a bottom end 16 and an outer wall 18 extending therebetween. The top end 14 tapers between a narrow side 20 and a wide side 22 such that the top end 14 has an ovoid shape. Thus, the top end 14 conforms to female genitalia thereby facilitating the top end 14 to capture urine for a urine sample. In this way the funnel 12 inhibits the urine from spilling onto a user's hands and clothing during the collection process of the urine sample. The bottom end 16 has a diameter that is less than a diameter of the top end 14.

The outer wall 18 has a front side 24 and a back side 26. The front side 24 extends in a straight line between the top end 14 and the bottom end 16 and the back side 26 curves rearwardly between the top end 14 and the bottom end 16. Thus, the top end 14 more efficiently conforms to the curvature and structure of the female's genitalia thereby reducing the likelihood of urine escaping the funnel 12.

A jar 28 is provided that has an upper end 30 and an outer wall 32. The upper end 30 is open and the outer wall 32 of the jar 28 is threaded adjacent to the upper end 30. A lid 34 is removably coupled to the jar 28 for closing the jar 28. The lid 34 has a top wall 36 and an outside wall 38 extending downwardly from the top wall 36. The outside wall 38 has an inwardly facing surface 40 and the inwardly facing surface 40 is threaded. The inwardly facing surface 40 threadably engages the outer wall 18 of the jar 28 having the top wall 36 abuts the upper end 30 of the jar 28. Moreover, the top wall 36 has an opening 42 extending therethrough.

A receiver 44 is coupled to the lid 34 and the bottom end 16 of the funnel 12 is removably insertable into the receiver 44. In this way the receiver 44 directs the urine into the jar 28 for the urine sample. The receiver 44 is aligned with the opening 42 and the receiver 44 has a distal end 46 with respect to the lid 34. The distal end 46 is open, the receiver 44 is hollow and the distal end 46 receives the bottom end 16 of the funnel 12. A cap 48 is provided and the cap 48 is threadably coupled to the jar 28 to close the jar 28 when the urine has been collected in the jar 28.

In use, the lid 34 is coupled to the jar 28 and the bottom end 16 of the funnel 12 is inserted into the distal end 46 of the receiver 44. The top end 14 of the funnel 12 is positioned against the female's genitalia and the female urinates into the funnel 12. The funnel 12 directs the urine into the receiver 44 and the receiver 44 directs the urine in the jar 28. In this way the urine is collected from the female for the urine sample without having the urine contact the female's hands and clothing. The lid 34 is removed from the jar 28 and the cap 48 is positioned on the jar 28 when the urine has been collected in the jar 28. Thus, the jar 28 is closed the transportation to a laboratory or other facility to conduct testing on the urine sample.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A female urine collection assembly being configured to facilitate a female to provide a urine sample, said assembly comprising:
a funnel having a top end and a bottom end, said top end tapering between a narrow side and a wide side such that said top end has an ovoid shape wherein said top end is configured to conform to female genitalia thereby facilitating said top end to capture urine for a urine sample; and
a jar having said funnel being fluidly coupled thereto wherein said jar is configured to receive the urine for the urine sample.

2. The assembly according to claim 1, further comprising a lid being removably coupled to said jar for closing said jar, said lid having a top wall and an outside wall extending downwardly from said top wall, said outside wall having an inwardly facing surface, said inwardly facing surface being threaded, said inwardly facing surface threadably engaging said outer wall of said jar having said top wall abutting said upper end of said jar, said top wall having an opening extending therethrough.

3. The assembly according to claim 2, further comprising a receiver being coupled to said lid, said bottom end of said funnel being removably insertable into said receiver wherein said jar is configured to receive the urine for the urine sample.

4. The assembly according to claim 3, wherein said receiver is aligned with said opening, said receiver having a distal end with respect to said lid, said distal end being open, said receiver being hollow, said distal end receiving said bottom end of said funnel.

5. A female urine collection assembly being configured to facilitate a female to provide a urine sample, said assembly comprising:
a funnel having a top end and a bottom end, said top end tapering between a narrow side and a wide side such that said top end has an ovoid shape wherein said top end is configured to conform to female genitalia thereby facilitating said top end to capture urine for a urine sample, said bottom end having a diameter being less than a diameter of said top end;
a jar having an upper end and an outer wall, said upper end being open, said outer wall being threaded adjacent to said upper end;
a lid being removably coupled to said jar for closing said jar, said lid having a top wall and an outside wall extending downwardly from said top wall, said outside wall having an inwardly facing surface, said inwardly facing surface being threaded, said inwardly facing surface threadably engaging said outer wall of said jar having said top wall abutting said upper end of said jar, said top wall having an opening extending therethrough; and
a receiver being coupled to said lid, said bottom end of said funnel being removably insertable into said receiver wherein said jar is configured to receive the urine for the urine sample, said receiver being aligned with said opening, said receiver having a distal end with respect to said lid, said distal end being open, said receiver being hollow, said distal end receiving said bottom end of said funnel.
